(19) United States
(12) Patent Application Publication
(10) Pub. No.: US 2004/0162545 A1
(43) Pub. Date: Aug. 19, 2004

(54) Bypass for glaucoma drainage device

(76) Inventors: J. David Brown, St. Paul, MN (US); Tingrui Pan, St. Paul, MN (US); Babak Ziaie, St. Paul, MN (US)

Correspondence Address: Schwegman, Lundberg, Woessner & Kluth, P.A., P.O. Box 2938, Minneapolis, MN, 55402, US

(21) Appl. No.: 10/664,409

(22) Filed: Sep. 16, 2003

Related U.S. Application Data
(60) Provisional application No. 60/448,311, filed on Feb. 14, 2003.

(51) Int. Cl. A61M 1/00

(52) U.S. Cl. 604/541

(57) ABSTRACT

Aqueous humor flow control for managing intraocular pressure in an eye. Excessive pressure due to formation of a fibrous capsule and valve resistance is relieved by bypassing the valve element or by providing a secondary discharge port. Removal of resistance is enabled by physical manipulation, external stimulus, chemical action or biological action. A resistor inserted in an intake conduit provides a predetermined resistance to flow and thus, a desired intraocular pressure.

### RELATED APPLICATIONS

This document claims priority, and is related to, commonly assigned U.S. Provisional Patent Application Serial No. 60/448,311, entitled “BYPASS FOR VALVED GLAUCOMA DRAINAGE DEVICE,” applicants Babak Ziaie, J. David Brown and Tingrui Pan, filed Feb. 14, 2003, the specification of which is hereby incorporated by reference in its entirety.

### GOVERNMENT FUNDING

This work is supported, at least in part, by the National Science Foundation, Agency Grant Number BES-0093604; University CUFS Number 522-6459. The United States government may have certain rights in the disclosed subject matter.

### TECHNICAL FIELD

This document relates generally to a glaucoma drainage device, and in particular, but not by way of limitation, to structures and methods for reducing intraocular pressure associated with a glaucoma drainage device.

### BACKGROUND

Glaucoma is currently the leading cause of irreversible blindness in the world. In the USA, millions of people suffer from glaucoma. Enormous amounts of money are spent on glaucoma treatment annually in the United States of America.

Elevated intraocular pressure is the outstanding risk factor for the development of glaucoma, and the main reason for progression of the disease. Recent randomized clinical trials have shown that glaucoma progression is halted only when intraocular pressure is lowered to extremely low levels, in the 8-12 mmHg range. Previously, intraocular pressures below 21 mmHg were considered normal, and safe, however, that is no longer the case.

Current glaucoma treatments include medicines, lasers, and surgery. Neither medicines nor lasers can consistently, or predictably, lower the IOP to the required levels. They also are temporary and expensive treatments. Surgical options include trabeculectomy and glaucoma drainage devices. Mitomycin C, an anti-fibroblastic drug, must be used with a trabeculectomy to allow the IOP to reach low enough levels. But, this drug has significantly added to the risks and complications of such filtering surgery. Mitomycin C causes thinning of the conjunctiva, which can lead to leaking, hypotony, and intraocular infections.

Glaucoma drainage devices consist of a tube shunting aqueous humor from the anterior chamber of the eye to an external sub-conjunctival plate made of synthetic biomaterials. Molteno, in 1969, described the first glaucoma drainage devices. The use of these early glaucoma drainage devices was limited by the frequent and often serious complications associated with the hypotony that occurred in the early postoperative period, before a fibrous capsule could form around the external plate to provide resistance to aqueous humor outflow. In 1993, Ahmed added a valve to a glaucoma drainage devices to address the problem of early postoperative hypotony. The valve provides a resistance to aqueous humor outflow prior to formation of the fibrous capsule, typically in 2-3 months.

Despite these developments in glaucoma drainage devices, elevated intraocular pressure continues to be a problem.

### SUMMARY

The present subject matter includes methods and systems for reducing the resistance to flow in a glaucoma drainage device. In one embodiment, the resistance is reduced by bypassing the valve in an implanted drainage device. In one embodiment, a drainage device operates in two modes with a greater flow resistance in a first mode and a lower flow resistance in a second mode. In various embodiments, multiple discharge ports, resistance elements, plugs, valves and controllable elements are configured to yield the two modes of operation. In one embodiment, a resistor disposed in an intake conduit provides a predetermined resistance to flow and thus, a desired intraocular pressure.

### BRIEF DESCRIPTION OF THE DRAWINGS

In the drawings, like numerals describe substantially similar components throughout the several views. Like numerals having different letter suffixes represent different instances of substantially similar components.

FIG. 1 illustrates an implantable drainage device and a linear element.

FIG. 2 illustrates an eye having a linear element within an implanted glaucoma drainage device.

FIG. 3 illustrates an exploded view of a drainage device with a valve assembly.

FIG. 4 illustrates a view of an elastic membrane of a valve assembly.

FIG. 5 illustrates a tubular linear element and an intake conduit.

FIG. 6 illustrates a valve assembly with a linear element.

FIG. 7 illustrates a valve with a linear element having a flange, retention barbs and a plurality of holes.

FIG. 8 illustrates solid linear element and an intake conduit.

FIG. 9 illustrates a view of a solid linear element bypassing a valve.

FIG. 10 illustrates a porous linear element.

FIG. 11 illustrates a laser light source for use with a membrane valve.

FIG. 12 illustrates a catheter cutter tool for use with a membrane valve.

FIG. 13A illustrates a valve having a bypass line.

FIG. 13B illustrates a resistive element in an intake conduit of a drainage device.

FIG. 13C illustrates a drainage device having a biodegradable valve member.

FIG. 14 illustrates a resistive element and a valve.

FIG. 15 illustrates a pair of resistive elements in a drainage device.

FIGS. 16 and 17 illustrate resistive elements in a tube.

FIG. 18 illustrates a gold impregnated resistive element.

FIG. 19 illustrates a porous resistive element.

FIG. 20 illustrates a ferromagnetic resistive element.

FIG. 21 illustrates a multi-bore resistive element.

FIG. 22 illustrates a resistive element with a gold membrane.

FIG. 23 illustrates a flow chart of a method according to one embodiment.

FIG. 24 illustrates a flow resistor disposed in the lumen of a tube.

### DETAILED DESCRIPTION

In the following detailed description, reference is made to the accompanying drawings that form a part hereof, and in which is shown, by way of illustration, specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the scope of the present subject matter. The following detailed description is, therefore, not to be taken in a limiting sense, and the scope of the present invention is defined by the appended claims and their equivalents.

The present subject matter relates to reducing a resistance through a glaucoma drainage device in order to produce a reduced intraocular pressure.

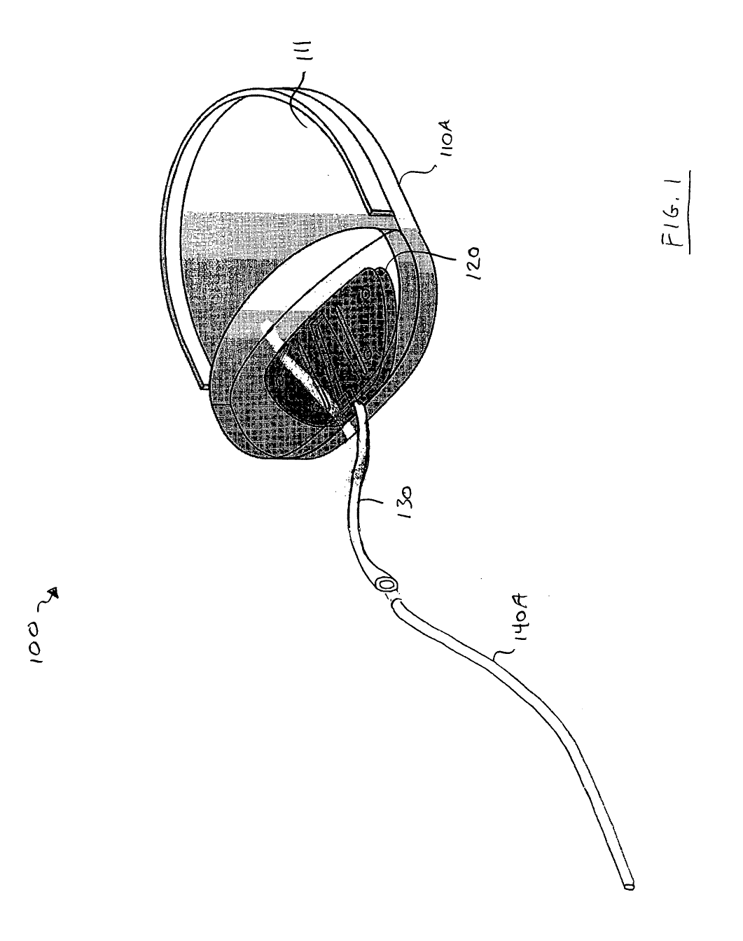

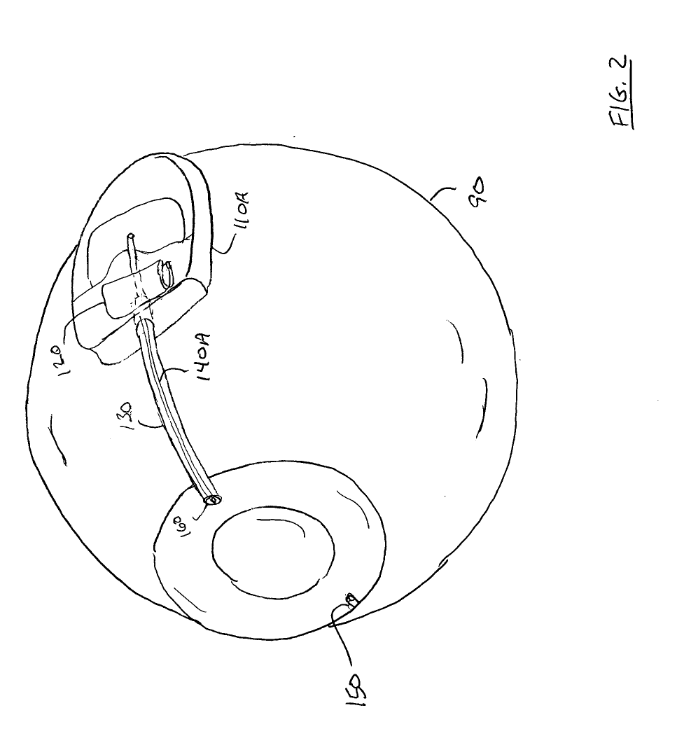

System 100 illustrated in FIG. 1 includes implantable glaucoma drainage device 110A having valve assembly 120 and intake conduit 130. Drainage device 110A, valve assembly 120 and intake conduit 130 are shown to be transparent for clarity purposes however, opaque materials are also contemplated. When implanted in a patient, as shown in FIG. 2, end 160 of intake conduit 130 is positioned in the anterior chamber of eye 90. The aqueous humor at the anterior chamber then flows through intake conduit 130, through valve assembly 120 and out on to the surface of external plate 111. Drainage device 110A is typically fabricated of biocompatible materials and is sometimes referred to as a valved glaucoma drainage device.

Linear element 140A is inserted in the lumen of intake conduit 130 and positioned in a manner to bypass valve assembly 120. Linear element 140A, in one embodiment, includes a polyimide microtube. In various embodiments, linear element 140A includes other biomaterial such as silicone, polytetrafluoroethylene, polypropylene, polymethyl methacrylate, acrylic, polyurethane, silastic, and metal.

Incision is made at 150 to enable placement of linear element 140A into intake conduit 130. Other incisions may be made to facilitate placement of the linear element.

FIG. 3 illustrates an exploded view of device 110B according to one embodiment of the present subject matter. Valve assembly 120 includes folded elastic membrane 122, cover plate 80 and lower support 45. An underside of cover plate 80 includes channel 70 and splines 65. Channel 70 provides relief to allow movement of elastic membrane 122.

Elastic membrane 122 is coupled to end 170 of intake conduit 130. End 160 of intake conduit 130 is open and receives the aqueous humor from the anterior chamber of the eye. Leaves 55 of elastic membrane 122 are modulated with changes in pressure.

Lower support 45 includes a plurality of pins 50. Holes 75 in cover plate 80 are configured to align with holes 60 in elastic membrane 122 and pins 50. In addition, lower support 45 includes keyways 40 to receive splines 65. The combination of splines 65, keyways 40, pins 50 and holes 75 serve to hold elastic membrane 122 in a taut position. A chamber formed by relief 70 and relief 35 allows movement of elastic membrane 122 and groove 30 receives intake conduit 130. Fluid discharged from valve assembly 120 is distributed on a surface of external plate 111.

Other valve configurations are also contemplated. For example, in one embodiment, rather than a folded elastic membrane, the valve includes a cruciate opening along the lumen of an intake conduit.

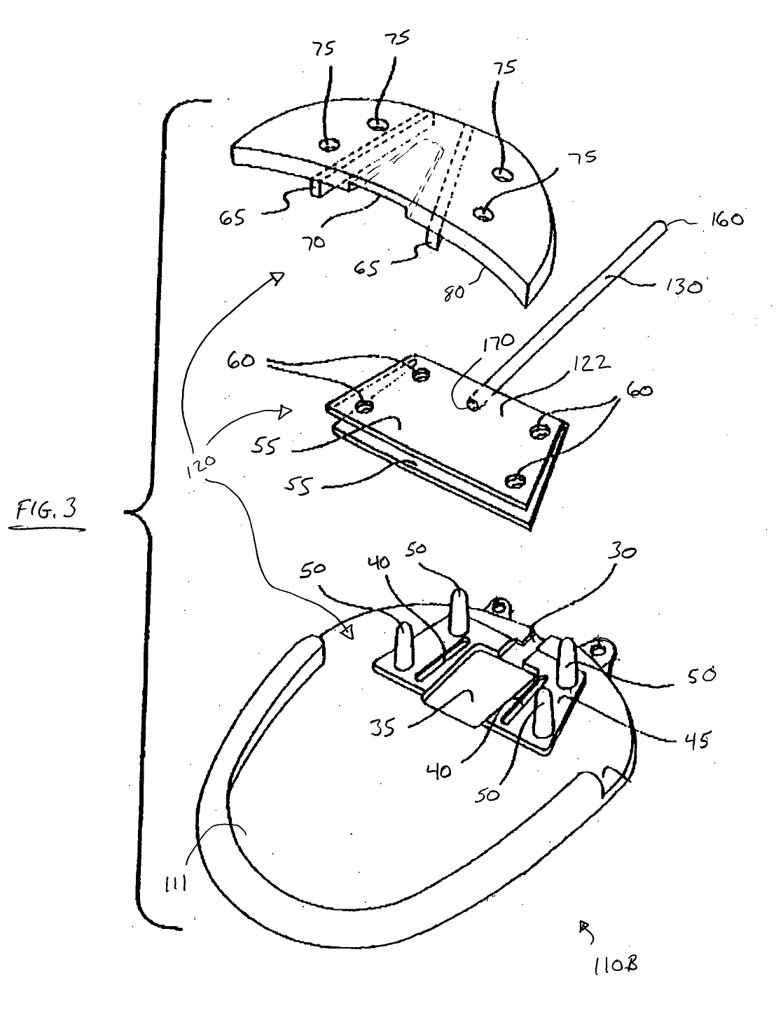

An elevation view of portions of valve assembly 120 is presented in FIG. 4. In the figure, solid lines depict elastic membrane 122 in an open position and the dashed lines are used to denote the closed position. As intraocular pressure rises, elastic membrane 122 opens to allow discharge of aqueous humor onto plate 110. When intraocular pressure drops, elastic membrane 122 closes to prevent any backflow to the anterior chamber. According to one embodiment, tube 130 includes silicone tubing.

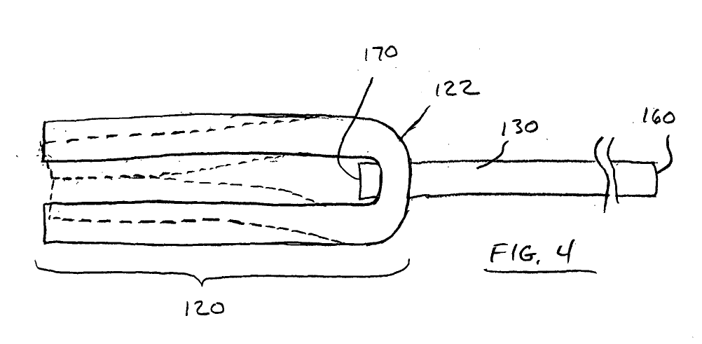

To reduce the flow resistance arising from the action of the valve assembly, according to one embodiment, a linear element is inserted into the lumen of the intake conduit. FIG. 5 illustrates linear element 140A relative to end 160 of intake conduit 130. Linear element 140A, in embodiment includes a hollow tube, which provides a bypasses for fluid traversing valve assembly 120.

FIG. 6 illustrates placement of linear element 140A through leaves 55 of elastic membrane 122. As shown in the figure, end 180A of linear element 140A is inserted into valve assembly 120 sufficiently far to prevent complete closure of elastic membrane 122. In addition, the lumen of linear element 140A provides a channel by which aqueous humor is discharged without encountering the resistance to flow ordinarily presented by valve assembly 120. In one embodiment, a quantity of aqueous humor also flows in the space between the exterior wall of linear element 140A and the interior wall of intake conduit 130.

In one embodiment, end 180A of the linear element is stabilized in a desired position. For example, according to one embodiment, end 180A is positioned approximately 2 cm from end 160 of intake conduit 130. Placement can be pre-determined using a B-scan ultrasound or using slit lamp examination.

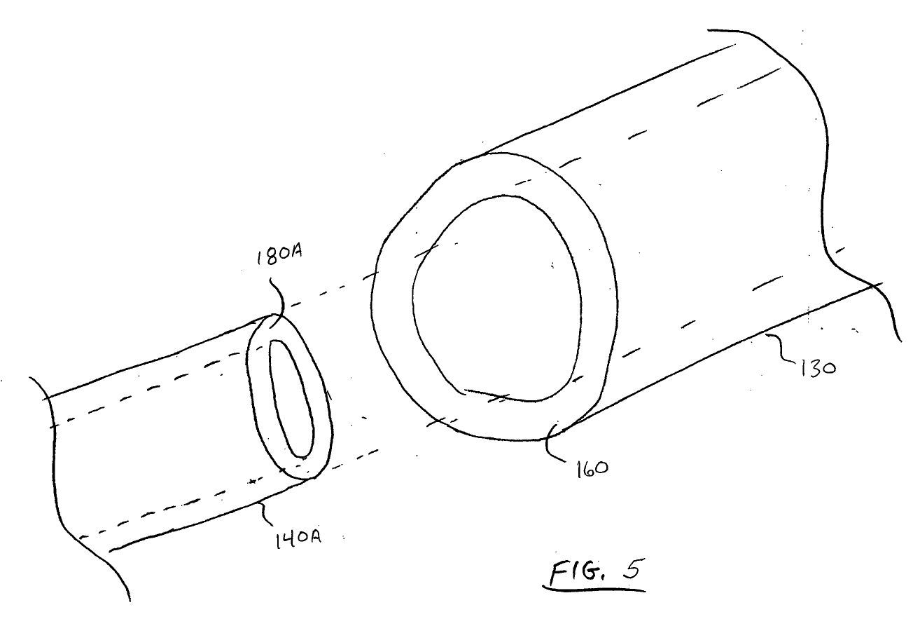

FIG. 7 illustrates one embodiment of the present subject matter. In the figure, intake conduit 130 and portions of valve assembly 120 are shown. Linear member 140B is disposed within the lumen of intake conduit 130 and displaces leaves 55 of elastic membrane 122. A plurality of barbs 185 are illustrated on the external surface of linear member 140B. The placement of barbs 185 are shown along the length of linear member 140B, however, in certain embodiments, barbs 185 are distributed in selected locations such as, near end 180B, in a central region, or near flange 195 disposed at an opposite end. Barbs 185 are configured to provide low resistance to insertion and high resistance to extraction of linear element 140B relative to intake conduit 130. In one embodiment, each barb 185 is angled posteriority. In one embodiment, each barb 185 includes a single fiber or shaft of material. In one embodiment, each barb 185 includes a circumferential skirt or rib formed on the external surface of linear element 140B.

In the embodiment shown, a plurality of holes 190 are distributed in the wall of linear element 140B. In one embodiment, a single hole 190 is provided. Hole 190 provides a discharge path for aqueous humor from within the lumen of linear element 140B to a region external to the lumen. In one embodiment, hole 190 is located in linear element 140B at a position near valve assembly 120 such that fluid in the lumen of linear element 140B is readily drained without encountering resistance presented by valve assembly 120.

In one embodiment, flange 195 is disposed at an end of linear element 140B. Flange 195 engages end 160 of intake conduit 130. In one embodiment, flange 195 includes a flared wall section. Flange 195 substantially limits the amount of aqueous humor permitted to flow in the space between the exterior of linear element 140B and lumen of intake conduit 130.

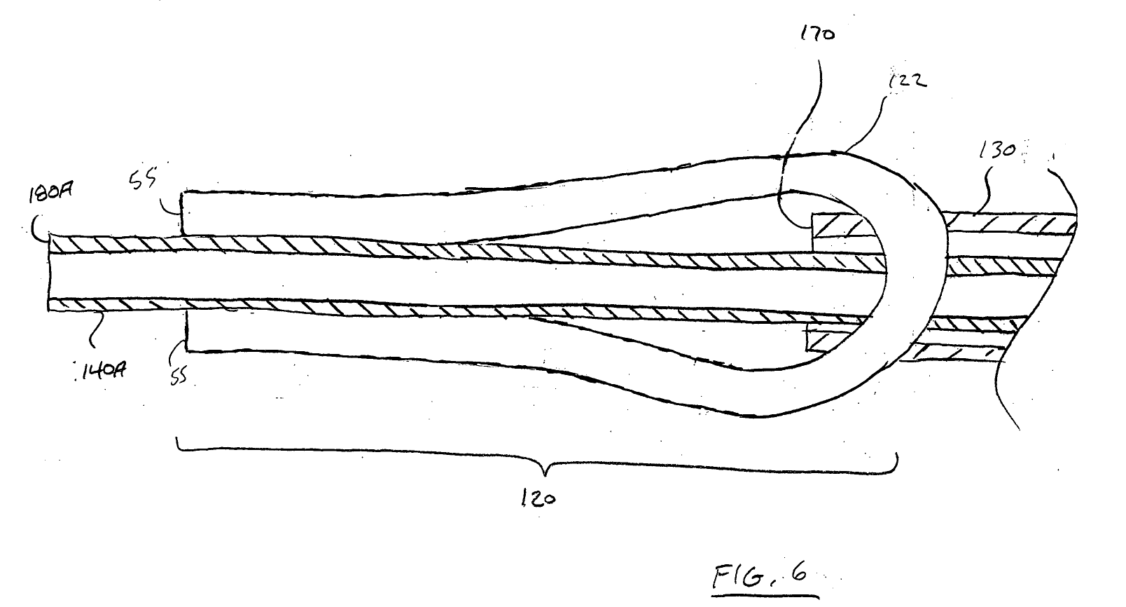

FIG. 8 illustrates a view of intake conduit 130 and linear element 140C having a solid section. Linear element 140C includes a segment of a rod having a round section. In addition to a round section, other configurations are also contemplated, including rectangular or square. Aqueous humor within intake conduit 130 is allowed to flow in the space between the external wall of linear element 140C and the lumen of intake conduit 130. FIG. 9 illustrates an axial view including end 170 of intake conduit 130 and end 180C of round solid linear element 140C. Linear element 140C is shown in an eccentric position and in contact with a lower portion of intake conduit 130. In one embodiment, linear element 140C includes barbs or skirts or other structures to stabilize the placement of linear element 140C relative to intake conduit 130. In one embodiment, linear element 140C is in concentric alignment with intake conduit 130. In the figure, elastic membrane 122 of valve assembly 120 is in contact with linear element 140C. Aqueous humor is permitted to freely flow from intake conduit 130 in the regions denoted as 162.

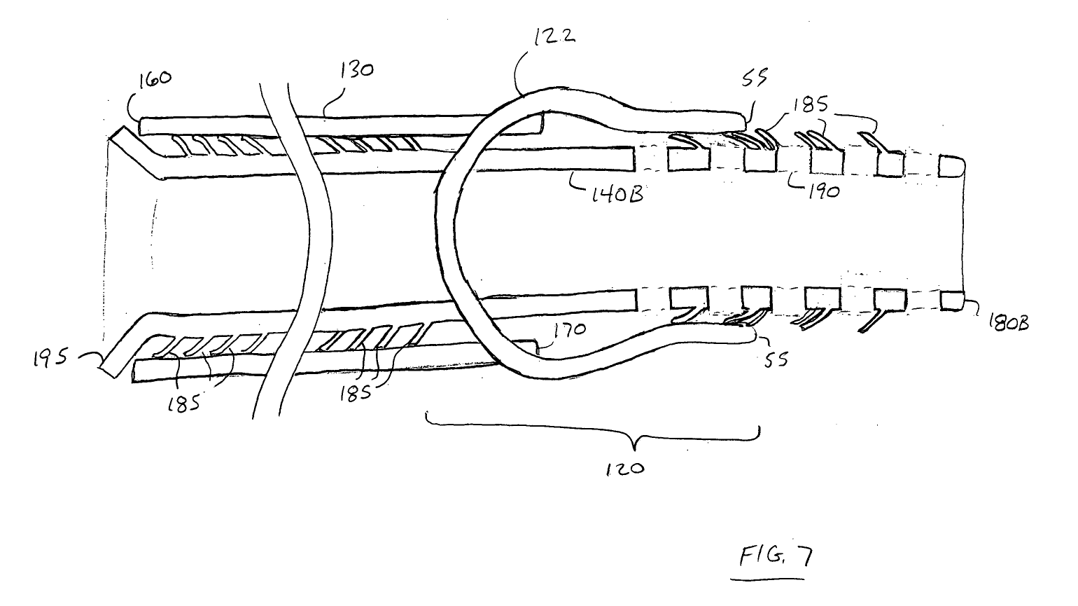

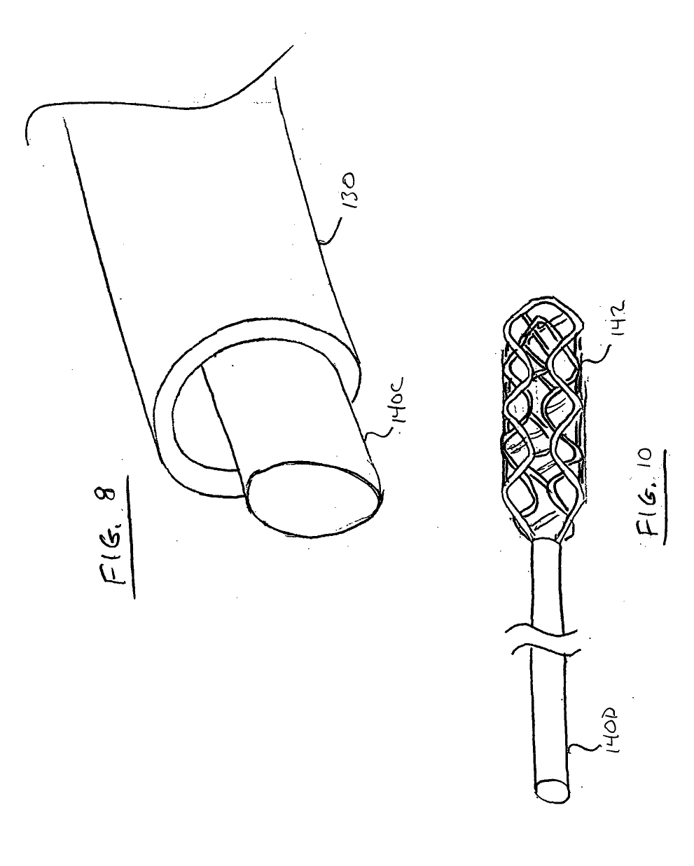

FIG. 10 illustrates porous segment 142 of linear element 140D. Porous segment 142, in one embodiment, is a shape memory material, such as a metal alloy. When at a first predetermined temperature, porous segment 142 is collapsed to a small diameter and when at a second temperature (typically, approximating that of a human body) porous segment 142 expands to a larger diameter as shown in the figure. Linear element 140D is inserted into intake conduit 130 and porous segment 142 is disposed at valve assembly 120. One method provides that porous segment 142 is cooled, or thermally soaked in a reduced temperature environment to cause contraction. In one embodiment, segment 142 collapses into a small diameter when cooled. At implantation, segment 142 is guided into intake conduit 130 and positioned in a manner that obstructs the movement of elastic membrane 122. When implanted in a body, segment 142 warms to body temperature and expands to a larger diameter, as shown in the figure, thus preventing complete closure of leaves 55.

In one embodiment, a portion of valve assembly 120 is removed to reduce resistance to flow of aqueous humor. FIG. 11 illustrates a laser light source 141 coupled to linear element 140E. In one embodiment, to reduce flow resistance, laser light emitted by linear element 140E is directed at elastic membrane 122, thereby ablating a portion of valve assembly 120. Residue from the removal process is captured and extracted or naturally flushed from the body. FIG. 12 illustrates a micro-catheter rotary cutter 142 within a sheath provided by linear element 140F. Cutter 142 is routed through intake conduit 130 and positioned at valve assembly 120. A protective sheath is retracted and cutter 142 removes portions of elastic membrane 122.

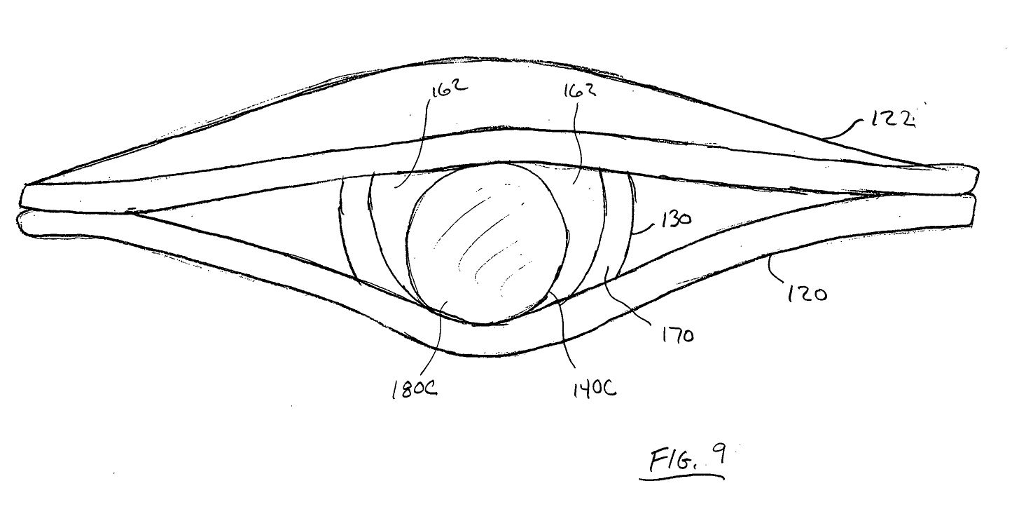

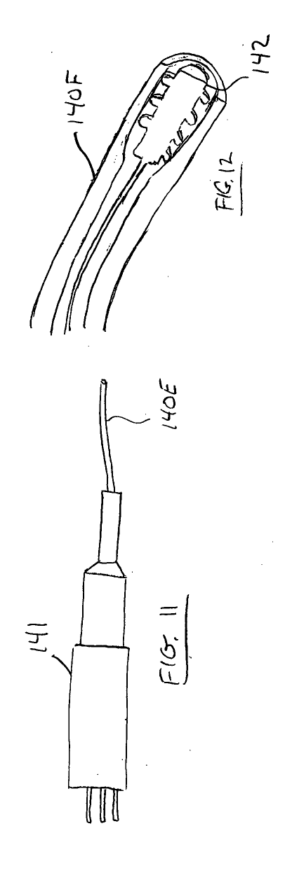

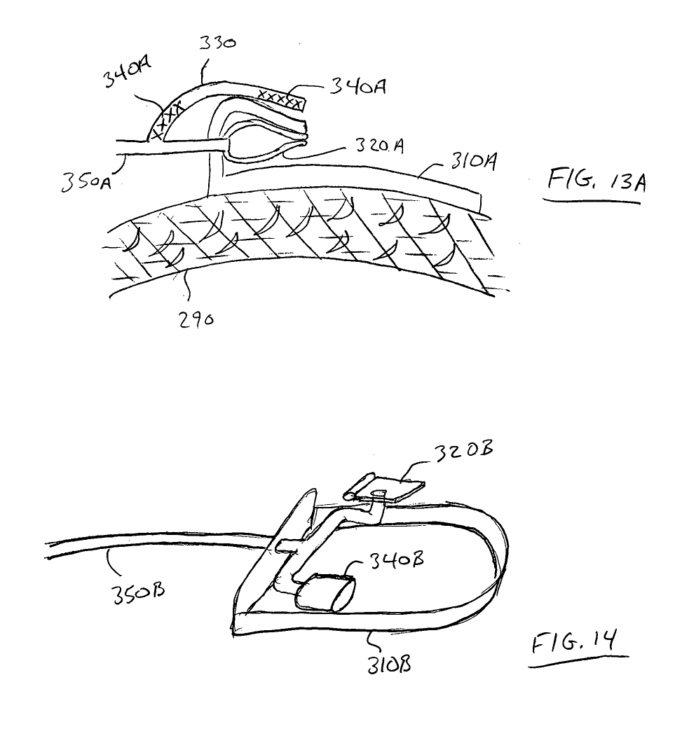

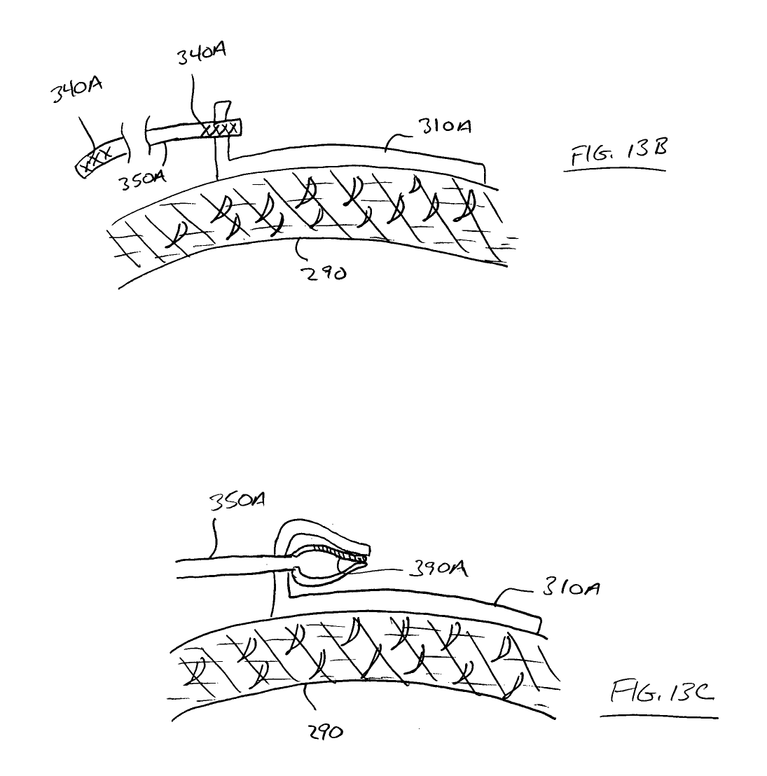

FIG. 13A illustrates one embodiment of a drainage device according to the present subject matter. In the figure, device 310A is disposed on a surface of sclera 290. Intake conduit 350A receives aqueous humor from the anterior chamber of the eye. Intake conduit 350A is bifurcated and with a first channel leading to valve assembly 320A and a second channel leading to bypass tube, or shunt 330. A portion of the lumen of shunt 330 includes resistor 340A. Resistor 340A presents a resistance to the flow of aqueous humor. In one embodiment, resistor 340A includes at least one plug which prevents the flow of aqueous humor.

At the time of implantation, and before formation of the fibrous capsule around device 310, aqueous humor received in intake conduit 350A is discharged by flowing through valve assembly 320A and resistor 340A blocks the flow of aqueous humor through shunt 330.

At some time after formation of the fibrous capsule, the resistance to flow through shunt 330 is selectively reduced or removed. For example, in one embodiment, resistor 340A includes a biodegradable polymer that dissolves and dissipates after a predetermined period of time. Examples of suitable polymers include, but are not limited to, polylactic acid (PLA), polyglycolic acid (PGA), poly lactide-co-glycolide (PLGA), polycaprolactone (PCL) and poly-1-lactic acid (PLLA).

The aqueous humor, like other liquids or currents, will follow the path of least resistance. Thus, when resistor 340A is removed (or its resistive value is reduced), all (or a larger portion) of the aqueous humor will flow through shunt 330 and none (or a reduced portion) of the aqueous humor flows through valve assembly 320A.

Shunt 330A discharges aqueous humor onto the surface of a plate of device 310A. In the figure, the bifurcation of intake conduit 350A is depicted at a point external to device 310A. In one embodiment, the bifurcation of the intake conduit occurs at a point on the interior of the drainage device.

In the embodiment shown, shunt 330A is illustrated routed above valve assembly 320A. Other placements of shunt 330A are also contemplated. For example, in various embodiments, shunt 330A is routed adjacent to valve assembly 320A or below valve assembly 320A. In one embodiment, shunt 330A is routed in a passage through sclera 290 and through a passage in a lower surface of device 310A.

FIG. 13B includes one embodiment of the present subject matter where intake conduit 350A does not shunt aqueous humor through a valve assembly but rather, the aqueous humor flows directly through a channel onto a surface of external plate 310A. In this embodiment, a portion of the lumen of intake conduit 350A is temporarily blocked with resistor 340A. At some time after formation of the fibrous capsule, the resistance to flow presented by resistor 340A is selectively reduced or removed. Resistor 340A, in various embodiments, is disposed at one or more positions within intake conduit 350A.

FIG. 13C includes one embodiment of the present subject matter where intake conduit 350A shunts aqueous humor through a valve assembly which includes biodegradable structure 390A. Biodegradable structure 390A forms all or a portion of the valve assembly. At some time after formation of the fibrous capsule, biodegradable structure 390A is naturally, or after stimulation, dissolved or disintegrated.

FIG. 14 illustrates intake conduit 350B coupled to device 310B having valve assembly 320B on a first branch line and resistive element 340B on a second branch line. In the figure, valve assembly 320B includes a cantilever structure, here shown as transparent, that opens to allow aqueous humor to discharge onto the plate. In addition, resistor 340B is shown coupled to the second branch of intake conduit 350B. Cover plate 80, along with selected other structure associated with valve assembly 320B, is omitted for clarity.

Valve assembly 320B, in one embodiment, includes a polymeric (silicone) cantilever valve. In one embodiment, the valve assembly includes a ball-type check valve. In one embodiment, valve assembly 320B opens at a predetermined intraocular pressure and is effective to prevent reflux of inflammatory blood cells or other pro-inflammatory or growth factor, into the anterior chamber.

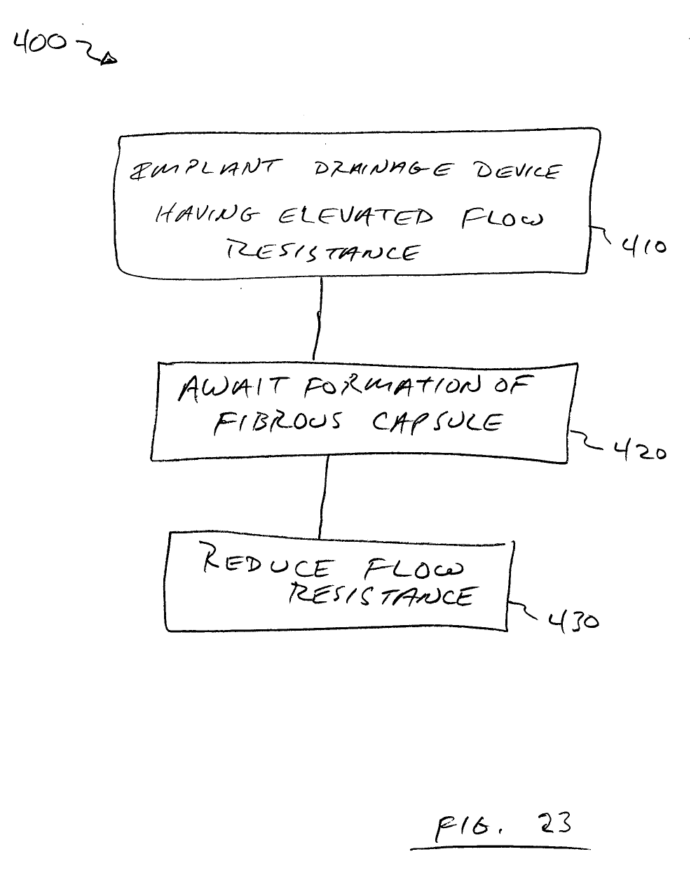

In one embodiment, rather than using a valve, the initial resistance is provided by a flow resistor having open channels or pores, as shown for example, in FIG. 21. The number, length, and size of the pores are selected to achieve a suitable resistance to generate a desired intraocular pressure. In one embodiment, pore size is selected sufficiently large to reduce likelihood of cellular blockage. A second outlet 340B is temporarily plugged. The positions of these two outlets, one to provide initial resistance and one temporarily plugged, in one embodiment, is shown in FIG. 14, however, other placements are also contemplated at, near, over, or within the external plate.

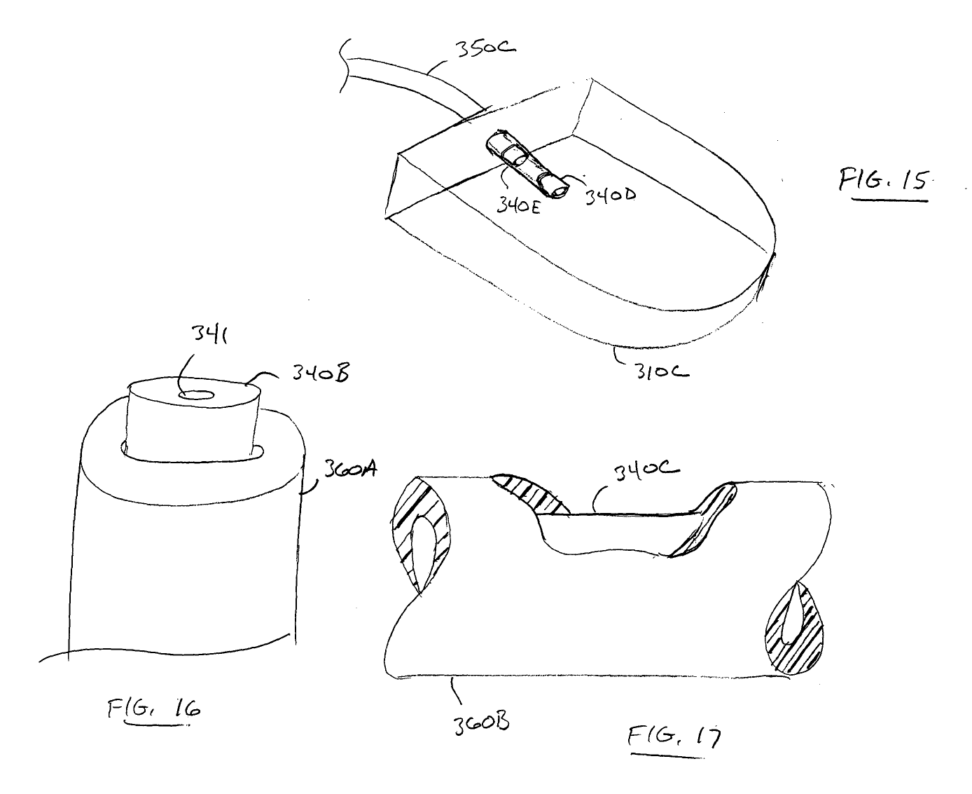

FIG. 15 illustrates intake conduit 350C coupled to device 310C having two or more series connected resistors 340E and 340D. In one embodiment, the combined resistance to flow presented by resistor 340E and resistor 340D is sufficient to prevent hypotony in the early postoperative period prior to formation of the fibrous capsule. At a later time, the combined resistance value presented by resistor 340E and resistor 340D is reduced. In one embodiment, the combined resistance is reduced by removing resistor 340D. In one embodiment, the combined resistance is reduced by removing resistor 340E. In one embodiment, both resistor 340D and resistor 340E are selectively removable. The selected resistance can be removed by physically extracting the element. The resistance can be reduced by degrading or dissolving portions of a selected resistor element by appropriate selection of materials and application of a stimulus as described elsewhere in this document.

FIG. 16 illustrates an embodiment of fluid resistor 340B in an end of a lumen of conduit, or tube, 360A. Fluid resistor 340B includes orifice 341. FIG. 17 illustrates an embodiment of fluid resistor 340C disposed in the length of a lumen of tube 360B. Resistors 340B and 340C, in various embodiments, includes a porous or multi-chamber element. In one embodiment, orifice 341 is omitted from resistor 340B.

FIG. 18 illustrates resistor 410 having a biodegradable polymer mixed with gold colloidal particles. In one embodiment, the particles include nano-particles or micro-particles. The resistance to flow can be reduced by removing all or a portion of resistor 410. The polymer can be removed by exciting the gold particles with external coil 415 placed in proximity to resistor 410. By exciting the gold particles, the temperature of the biodegradable polymer is increased above the polymer melting point. Coil 415 can be excited with a radio frequency field or other signal.

FIG. 19 illustrates resistor 420 having a porous or foamed biodegradable polymer. To reduce the resistance, external ultrasound unit 425 is used to excite and break down the polymer of resistor 420.

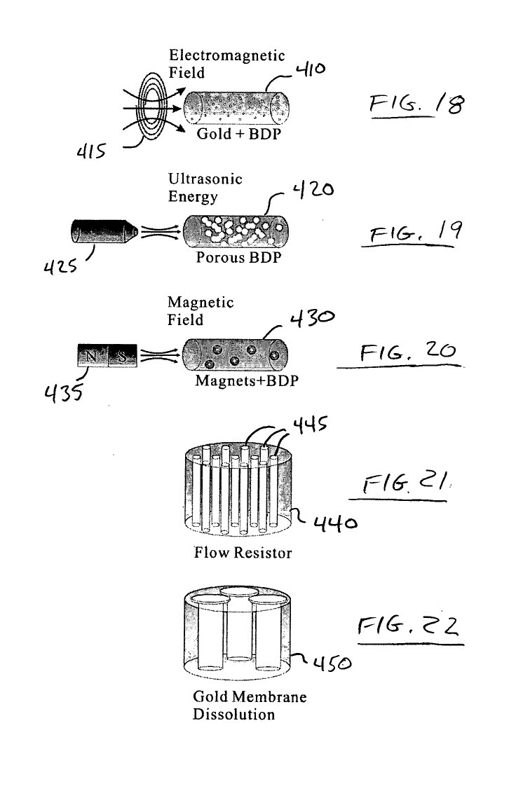

FIG. 20 illustrates resistor 430 having a mix of very small ferromagnetic particles within a biodegradable polymer. In one embodiment, externally applied magnet 435 is used to withdraw resistor 430 from a lumen. In one embodiment, externally applied magnet 435 provides a changing magnetic field that causes vibration or movement of the ferromagnetic particles. When vibrated or moved, the ferromagnetic particles generate heat which elevates the temperature of the polymer. At an elevated temperature, the polymer dissolves or biodegrades.

FIG. 21 illustrates flow resistor 440 having a plurality of bores or orifices 445 by which fluid is restrained. The numerosity, length and size of orifices 445 are selected to produce the desired resistance. Other types of flow resistors are also contemplated. For example, in one embodiment, a flow resistor includes a plurality of spherical beads with the bead size and numerosity selected for a desired resistance. In one embodiment, the beads include a polymer that dissolves, disintegrates or is otherwise selectively removable.

FIG. 22 illustrates resistor 450 having three orifices, each covered by a gold membrane. Embodiments with more or less than three orifices are also contemplated. Application of a telemetry derived DC voltage dissolves the gold membrane and thus reduces resistance to the flow of aqueous humor.

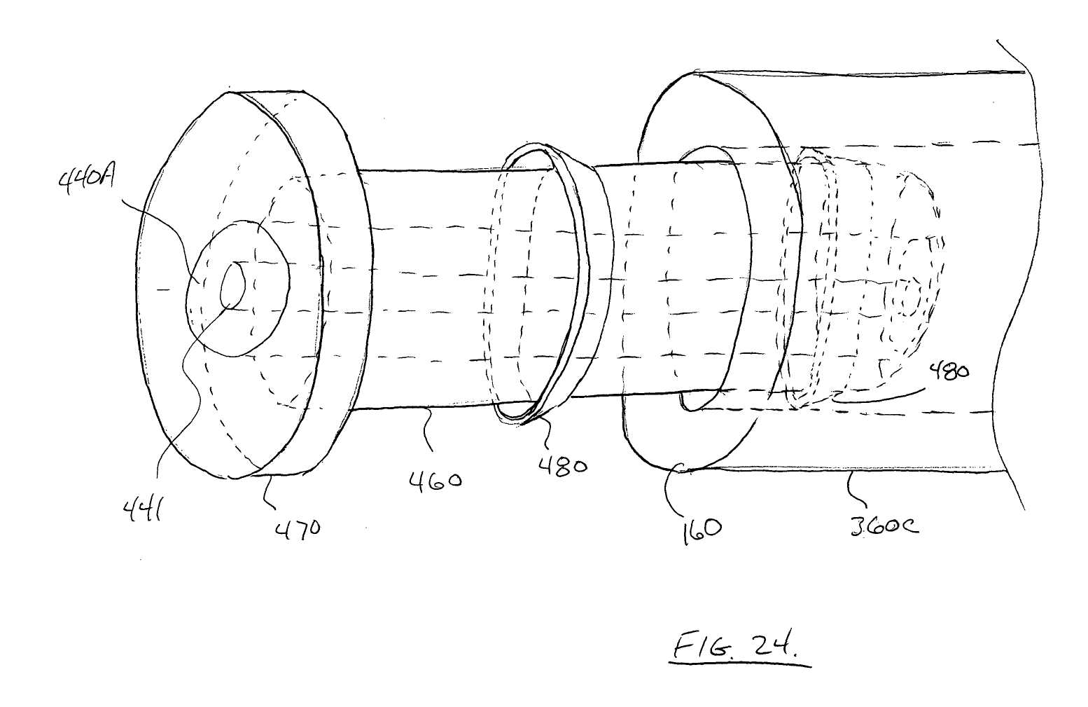

FIG. 23 illustrates method 400 according to one embodiment. At 410, a drainage device is implanted in a body. The drainage device is initially configured for high flow resistance. In various embodiments, a high flow resistance mode is presented by an elastic membrane of a valve assembly, a cantilever valve, a plug, a flow resistor or other structure.

At 420, the method includes awaiting the formation of the fibrous capsule. In various patients, the fibrous capsule may take a few weeks to a year to form, however, other time periods are also contemplated.

At 430, the flow resistance of the drainage device is reduced. In various embodiments, this entails bypassing an elastic membrane of a valve assembly, removing a portion of a valve assembly, removing a resistance, removing a plug, or by providing a bypass shunt line to increase the flow rate of aqueous humor. Various methods are available to stimulate the reduction in resistance. For example, application of an electric field, magnetic fields, ultrasound, a pH level, an enzymatic or hydrolytic degradation, or other stimulus may be applied.

In one embodiment, insertion of the linear element includes forming a small paracentesis incision in the cornea at a point opposite the opening of the intake conduit, followed by injection of a viscoelastic material. Through the paracentesis, a linear element is inserted into the intake conduit, as shown in FIG. 2. The linear element is inserted by visually observing progress. The linear element is routed across the anterior chamber and threaded into the lumen of the intake conduit. In one embodiment, the linear element is inserted to a distance of between approximately 1 mm and 1 cm beyond the valve assembly. In one embodiment, the intake tube is positioned within the superiortemporal quadrant and the linear member is inserted via a paracentesis within the inferior nasal quadrant. The linear member is inserted to a depth determined by the plate position. In one embodiment, the linear element length is determined by the plate position and the length of the intake conduit.

Portions of the structures presented in this document are fabricated of bioinert materials. In one embodiment, a surface coating including self-assembled monolayers (SAMs) of biomolecules is used. Examples of SAMs include phosphoryl choline, polyethylene oxide and polyethylene glycol and other materials that provide a hydrophilic surface, thereby decreasing or eliminating protein and cellular adhesion.

In one embodiment, the anterior chamber is filled by injecting a viscoelastic material. The linear element is threaded up the lumen of the intake conduit using normally available ocular surgical instruments and the linear element is positioned such that the leaves of the valve assembly are obstructed.

In one embodiment, the length of the linear member is selected prior to insertion in the intake conduit. In one embodiment, the length of the linear member is trimmed to size after insertion. In various embodiments, the intake end of the linear member extends beyond the end of intake conduit, terminates within the intake conduit or is flush with an end of the intake conduit.

In various embodiments, the linear member is fabricated of material including, polytetraflouethylene (PTFE), silicone, silastic, acrylic, polypropylene, polyimide or metal. The linear element material is selected to provide sufficient rigidity to allow insertion within intake conduit and within the leaves of valve assembly and to be flexible enough to follow the outer curve of the eye. The linear element is configured to have sufficient structural strength to hold the leaves of the valve assembly in an open position and to avoid significant compression of the linear member.

In one embodiment, the linear element includes a microstent or microtube.

### Alternative Embodiments

In one embodiment, one branch of an intake conduit is coupled to an adjustable resistor and another branch is coupled to a valve. In one embodiment, one branch of an intake conduit is coupled to an adjustable resistor and another branch is coupled to a fixed resistor.

In one embodiment, the resistance is infinite in that the resistance includes a plug.

In one embodiment, a single valve is provided in the implantable device. The valve is configured to present a desired resistance to fluid flow prior to formation of the fibrous capsule. Following formation of the fibrous capsule, the valve is removed, disabled or modified to present a reduced resistance to fluid flow. The valve is removed, disabled or modified using at least one of any combination of materials, methods and structures described herein.

In one embodiment, the drainage device includes a selectable member that allows operation in two or more modes, with each mode associated with a different resistance to fluid flow. For example, in one embodiment, a drainage device operates in a first mode having a low fluid flow resistance and a second mode having a high fluid flow resistance. The high fluid flow resistance is typically presented during the early post-operative time period and a low fluid flow resistance is typically presented during the later post-operative time period. In one embodiment, multiple modes are presented, with each mode associated with a different fluid flow resistance.

In one embodiment, a particular mode, and thus, a particular resistance value, is selected by applying an external stimulus. For example, in various embodiments, a radio frequency signal, a magnetic field, an optical signal, a temperature, an audio signal, an ultrasonic signal and other stimulus are used to select a mode having a lower resistance to flow. In one embodiment, a stimulus is applied to select a higher resistance to flow.

In one embodiment, an enzyme is introduced to the device to reduce the resistance. The enzyme, in one embodiment, includes an aqueous humor-borne enzyme. In one embodiment, hydrolytic degradation is used to change the resistance to fluid flow. In one embodiment, exposure to a predetermined pH level is used to trigger the change in resistance. In one embodiment, mechanical stimulation is used to change the resistance. In one embodiment, a biodegradable polymer is used and after a predetermined period of time, the polymer dissolves sufficiently to change the resistance.

One embodiment of the present subject matter provides that portions of valve assembly 120 are fabricated of materials that are removable or dissolvable. For example, and with respect to FIG. 3, in one embodiment, pins 50 are biodegradable or selectively removable. In one embodiment, splines 65 are biodegradable or selectively removable. In one embodiment, a portion of the elastic membrane is biodegradable or selectively removable.

In one embodiment, a remotely adjustable check-valve array includes an electrochemical release mechanism. An SU-8 polymer layer is deposited atop a gold sacrificial layer to form a valve structure. A constant DC current obtained via a telemetry link is used to electrochemically dissolve the gold sacrificial layer and activate the micromachined valves. The actuation mechanism is based on the electrochemical dissolution of a thin gold membrane which occurs through the formation of water-soluble chloro-gold (III) complexes in the saline solution. A microvalve array is fabricated using microelectromechanical system processes including chemical vapor deposition, lift-off, reactive ion etching and SU-8 photolithography. Activation by telemetry includes electronic circuitry for inductively receiving a wireless signal, rectifying the received signal and generating a DC current using a current source. Selected valves of the array are released to achieve a desired resistance to fluid flow.

In one embodiment, any combination of the length, the thickness and the stiffness of a cantilever microvalve is adjusted to achieve a desired resistance to fluid flow.

Under certain circumstances, it may be desirable to insert a resistor into the flow path of a drainage device. In one embodiment, a linear member is inserted into an intake conduit to provide a selected resistance to the flow of aqueous humor. FIG. 24 illustrates, for example and according to one embodiment, solid linear element 460, having surface barbs 480 and flange 470, placed in end 160 of intake conduit 360C. Linear element 460 includes a solid rod or plug, and effectively occludes the lumen of intake conduit 360C. In one embodiment, linear element 460 includes a flow resistor and provides resistance to flow without entirely occluding fluid flow. Linear element 460, in one embodiment, is fabricated of polyimide or other material as described elsewhere in this document.

Two barbs 480 are illustrated in the figure, each having a conical shape that engages the lumen of, and resists removal from, intake conduit 360C. In the figure, one barb 480 is illustrated in a deflected mode and another barb 480 is illustrated in a relaxed or un-deflected mode, however, more or less than two barbs are also contemplated. In addition, other structures to restrict retraction from the lumen are also contemplated. For example, filament type barbs, as shown in FIG. 7, helical structures, or other types of retention devices are also contemplated.

Linear member 460, in one embodiment, includes a biodegradable polymer, and provides either complete occlusion of the lumen or provides a predetermined resistance to flow. Linear device 460, in various embodiments, includes a plurality of bores, orifices or beads to provide a predetermined resistance to flow. In one embodiment, linear element 460 includes core 440A having central orifice 441. Central orifice 441 presents a first resistance to fluid flow. After degradation or removal of core 440A, a second flow resistance is presented. In one embodiment, multiple cores are provided in linear element 460 and each is selectively degradable or removable.

Intake conduit 360C, as with the other intake conduits described elsewhere in this document, is coupled to a drainage device having an external plate. The drainage device, according to one embodiment, is of a valveless type as shown in FIGS. 13B and 15. The drainage device, in various embodiments, is fabricated as a valveless device or rendered so. The drainage device, according to one embodiment, presents an effective flow resistance equal to that of the intake conduit itself.

### Conclusion

The above description is intended to be illustrative, and not restrictive. Many other embodiments will be apparent to those of skill in the art upon reviewing the above description.

What is claimed is:

1. A system comprising:
an implantable drainage device having a valve coupled to an intake conduit and an external plate, the valve providing a resistance to fluid flowing through the intake conduit; and
a linear member configured for insertion through the valve thereby reducing the resistance.

2. The system of claim 1 wherein the linear member includes a tube adapted to bypass the valve.

3. The system of claim 2 wherein the tube includes a flange at a first end.

4. The system of claim 2 wherein the tube includes a plurality of barbs on an exterior surface of the tube.

5. The system of claim 2 wherein the tube includes at least one hole in a wall.

6. The system of claim 1 wherein the linear member includes a shape memory material having a first configuration at a first temperature and a second configuration at a second temperature, wherein in the second configuration, the linear member is adapted to bypass the valve.

7. The system of claim 1 wherein the linear member includes a rod adapted to bypass the valve.

8. The system of claim 7 wherein the rod includes a plurality of barbs on an exterior surface.

9. The system of claim 7 wherein the rod is porous.

10. The system of claim 1 wherein the linear member includes at least one of any combination of polyimide, silicone, polytetrafluoroethylene, polypropylene, polymethyl methacrylate, acrylic, polyurethane, silastic and metal.

11. The system of claim 1 wherein the linear member includes at least one of any combination of a laser light source and a micro-catheter cutter.

12. An implantable device comprising:
a flange;
a tubular shaft concentrically affixed to a face of the flange; and
a plurality of barbs on an exterior surface of the tubular shaft, and
wherein the plurality of barbs are configured to stabilize the tubular shaft relative to an interior surface of an intake conduit of a drainage device wherein the tubular shaft bypasses a valve coupled to the intake conduit.

13. The device of claim 12 wherein the flange is adapted to engage an end of the intake conduit.

14. The device of claim 12 wherein the tubular shaft includes at least one of any combination of polyimide, silicone, polytetrafluoroethylene, polypropylene, polymethyl methacrylate, acrylic, polyurethane, silastic and metal.

15. The device of claim 12 wherein the plurality of barbs are disposed over a length of the tubular shaft.

16. The device of claim 12 further including at least one hole in a wall of the tubular shaft.

17. An implantable device comprising:
an intake conduit; and
a flow controller coupled to the intake conduit, the flow controller having a first mode that provides a first resistance to a fluid entering the intake conduit and a second mode that provides a second resistance to the fluid and wherein the second mode is remotely selectable; and
wherein the second resistance is less than the first resistance.

18. The device of claim 17 wherein the fluid drains from the flow controller at a first discharge port when in the first mode and at a second discharge port when in the second mode.

19. The device of claim 18 wherein the first discharge port differs from the second discharge port.

20. The device of claim 17 wherein the flow controller includes a bypass port that presents a high flow resistance in the first mode and a low flow resistance in the second mode.

21. The device of claim 20 wherein the bypass port includes a discharge conduit coupled to the flow controller.

22. The device of claim 17 wherein the flow controller includes a biodegradable polymer.

23. The device of claim 22 wherein the biodegradable polymer is porous or foamed.

24. The device of claim 22 wherein the biodegradable polymer includes at least one of any combination of polylactic acid, polyglycolic acid, poly lactide-co-glycolide, polycaprolactone, and poly-1-lactic acid.

25. The device of claim 17 wherein the flow controller includes an enzymatic or hydrolytic degradable material.

26. The device of claim 17 wherein the flow controller includes a material sensitive to at least one of any combination of a predetermined pH, a predetermined magnetic field, a predetermined ultrasonic signal, a predetermined electric field, a predetermined DC current, a predetermined temperature, a predetermined light signal and a predetermined mechanical stimulation.

27. The device of claim 17 wherein the flow controller includes a polymer having at least one of any combination of gold colloidal particles and ferromagnetic particles.

28. The device of claim 17 wherein the flow controller includes a gold membrane.

29. The device of claim 17 wherein the flow controller includes a first valve.

30. The device of claim 29 further wherein the flow controller includes a second valve and wherein,
when in the first mode, the second valve is in a closed position and,
when in the second mode, the second valve is in an open position.

31. The device of claim 29 further wherein the flow controller includes a flow resistor having a selectable flow resistance and wherein,
when in the first mode, the flow resistor provides a high flow resistance and,
when in the second mode, the flow resistor provides a low flow resistance.

32. The device of claim 31 wherein the flow resistor includes a plug.

33. The device of claim 17 wherein the flow controller includes a first flow resistor.

34. The device of claim 33 wherein the first flow resistor includes a plurality of channels.

35. The device of claim 33 wherein the first flow resistor is porous.

36. The device of claim 33 further wherein the flow controller includes a second flow resistor having a selectable flow resistance and wherein,
when in the first mode, the second flow resistor provides a high flow resistance and,
when in the second mode, the second flow resistor provides a low flow resistance.

37. The device of claim 36 wherein the second flow resistor includes a plug.

38. The device of claim 33 wherein,
when in the first mode, the first flow resistor provides a high flow resistance and,
when in the second mode, the flow controller provides a low flow resistance.

39. The device of claim 29 wherein the second mode is selected when the first valve is maintained in an open position.

40. The device of claim 29 wherein the second mode is selected when at least a portion of the first valve is removed.

41. The device of claim 29 wherein the first valve includes an elastic membrane.

42. The device of claim 29 wherein the first valve includes an elastic membrane subjected to a first tension and wherein the second mode is selected when the elastic membrane is subjected to a second tension different from the first tension.

43. The device of claim 41 further including a membrane support configured to exert a first tension on the elastic membrane and wherein the second mode is selected when a portion of the membrane support is removed.

44. The device of claim 43 wherein the membrane support includes a plurality of pins and wherein the portion includes at least one pin of the plurality of pins.

45. The device of claim 17 wherein the flow controller includes an electromagnetically sensitive valve.

46. The device of claim 17 wherein the flow controller includes flow resistor sensitive to an electromagnetic signal or an electrochemical signal.

47. The device of claim 17 wherein the flow controller includes a material that is weakened upon exposure to a predetermined magnitude of at least one of any combination of electromagnetic stimulus, ultrasonic stimulation, magnetic stimulation, electric field stimulation, a temperature, light or mechanical stimulation.

48. The device of claim 17 wherein the flow controller includes a material that is weakened upon exposure to at least one of any combination of a predetermined level of pH, a predetermined enzyme and a predetermined energy level.

49. The device of claim 17 wherein the flow controller includes a material that is weakened upon exposure to a biological environment after a predetermined period of time.

50. A method comprising:
inserting a linear member in an intake tube of an implantable drainage device, the intake tube coupled to a valve and an external plate, the valve providing a resistance to fluid flowing through the intake conduit; and
positioning the linear member in a manner to reduce the resistance presented by the valve.

51. The method of claim 50 wherein positioning the linear member includes bypassing the valve.

52. The method of claim 51 further including engaging a plurality of barbs on a surface of the linear member with a lumen of the intake tube.

53. The method of claim 51 further including engaging a flange on the linear member with an orifice of the intake tube.

54. The method of claim 50 wherein positioning the linear member includes:
manipulating a laser light source; and
ablating a portion of the elastic membrane.

55. The method of claim 50 wherein positioning the linear member includes:
manipulating a mechanical cutter; and
removing a portion of the elastic membrane with the cutter.

56. The method of claim 50 further including thermally soaking the linear member at a first predetermined temperature; and
wherein positioning the linear member includes:
maneuvering the linear member into a position proximate the valve while at a temperature approximately that of the first predetermined temperature; and
changing a shape of the linear member upon exposure to a second predetermined temperature, wherein the first predetermined temperature differs from the second predetermined temperature.

57. A method comprising:
providing an implantable conduit for fluid drainage, the conduit having a first resistance to fluid flow in a first mode and a second resistance to fluid flow in a second mode, the first resistance greater than the second resistance; and
exposing the conduit to a stimulus wherein the stimulus is tailored to transition the conduit from the first mode to a second mode.

58. The method of claim 57 wherein exposing the conduit to the stimulus includes exposing the conduit to at least one of any combination of an electric field, a DC current, a magnetic field, an ultrasonic signal, an enzyme, hydrolysis, a pH level, a predetermined temperature, a light signal, a mechanical stimulation and a biological fluid.

59. The method of claim 57 wherein exposing the conduit to the stimulus includes opening a valve or changing a resistance of a fluid resistor.

60. The method of claim 59 wherein changing the resistance of the fluid resistor includes removing a plug.

61. A system comprising:
a drainage device having an intake conduit coupled to an external plate; and
a flow resistor adapted for insertion in a lumen of the intake conduit.

62. The system of claim 61 wherein the drainage device includes at least one of a valveless glaucoma drainage device, a glaucoma drainage device having a bypassed valve, a glaucoma drainage device having a disintegratable valve and a glaucoma drainage device having a dissolvable valve.

63. The system of claim 61 wherein the flow resistor includes at least one barb on an external surface.

64. The system of claim 61 wherein the flow resistor includes a plurality of beads.

65. The system of claim 61 wherein the flow resistor includes at least one bore.

66. The system of claim 61 wherein the flow resistor includes a biodegradable polymer.

67. The system of claim 61 wherein the flow resistor is remotely removable.

68. The system of claim 61 wherein the flow resistor includes a plug.

69. A device comprising:
a flow resistor adapted for placement in a lumen of an intake conduit of an implantable drainage device; and
at least one barb on an external surface of the flow resistor.

70. The device of claim 69 wherein the flow resistor includes a plurality of beads.

71. The device of claim 69 wherein the flow resistor includes at least one bore.

72. The device of claim 69 wherein the flow resistor includes a biodegradable polymer.

73. The device of claim 69 wherein the flow resistor is remotely removable.

74. The device of claim 69 wherein the flow resistor includes a plug.

75. The device of claim 69 wherein the flow resistor includes at least one of any combination of polyimide, silicone, polytetrafluoroethylene, polypropylene, polymethyl methacrylate, acrylic, polyurethane, silastic and metal.
